(19) United States
(12) Patent Application Publication
(10) Pub. No.: US 2025/0130191 A1
(43) Pub. Date: Apr. 24, 2025

(54) ADDITIVE MANUFACTURING OF CONDUCTING POLYMER ELECTRONICS WITH BIOACTIVE SUBSTRATES AND ENCAPSULATIONS AND RELATED DEVICES

(71) Applicant: Washington University, St. Louis, MO (US)

(72) Inventors: Alexandra Rutz, St. Louis, MO (US); Tianran Liu, St. Louis, MO (US)

(73) Assignee: Washington University, St. Louis, MO (US)

(21) Appl. No.: 18/922,171

(22) Filed: Oct. 21, 2024

Related U.S. Application Data
(60) Provisional application No. 63/591,927, filed on Oct. 20, 2023.

(51) Int. Cl. G01N 27/02; B29C 64/112; B29C 64/40; B29K 25/00; B29K 81/00; B29L 31/00; B33Y 10/00; B33Y 40/20; B33Y 80/00; G01N 33/483

(52) U.S. Cl. CPC G01N 27/026; B29C 64/112; B29C 64/40; B33Y 10/00; B33Y 40/20; B33Y 80/00; G01N 33/4833; B29K 2025/00; B29K 2081/04; B29K 2995/0005; B29L 2031/753

(57) ABSTRACT

Among the various aspects of the present disclosure are the provision of a bioelectronic device, additive manufacturing methods of bioactive encapsulated conducting polymer hydrogel electrodes, and related methods of use. As described herein, a 3D-printed bioelectronic electrode device, methods to fabricate a bioelectronic device, a method to perform a bioelectronics experiment, and a method to treat a subject with a bioelectronic-related disorder are described.

### CROSS-REFERENCE TO RELATED APPLICATIONS

This application claims priority from U.S. Provisional Application Ser. No. 63/591,927 filed on Oct. 20, 2023, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to bioelectronic devices, additive manufacturing methods of conducting polymer hydrogel electronics with bioactive encapsulations, and related methods of use.

### BACKGROUND OF THE INVENTION

Electrodes enable the communication between electrical systems and biological systems by applying stimuli or recording signals. As an indispensable tool of modern biology and medicine, electrodes are essential for advancing the understanding of cellular and tissue-level communication, promoting regeneration, regulating bioelectrical processes, and monitoring patient well-being. Commercially available electrodes are mainly composed of inorganic conductors and semiconductors, such as platinum and silicon, insulated with plastic encapsulation materials. These electrodes are dry, rigid, and not recognized by biological systems as a part of itself. Such material properties can lead to issues including but not limited to: lack of cell attachment and different cell behaviors in vitro, exacerbated foreign body reaction, fibrotic encapsulation, and damage to the surrounding tissues in vivo.

To improve device integration with biological systems, one strategy is to utilize soft and hydrated materials as electrode components to present a tissue-like microenvironment. Conducting polymers such as poly(3,4-ethylenedioxythiophene) polystyrene sulfonate (PEDOT:PSS) can form hydrogels with water fraction and stiffness that are comparable to soft tissues. The commercial availability of PEDOT:PSS as an aqueous colloidal dispersion in addition to its high conductivity as a hydrogel make PEDOT:PSS a great candidate for the conducting component of a cyto-compatible and bio-compatible electrode.

In addition to the electrode, another important device component to consider is the encapsulation. As the outermost layer of an electrode array, the encapsulation layers constitute the majority of the device mass and surface area, thus significantly impacting the device's compatibility with biological systems. To further promote cell attachment and tissue regeneration, materials that actively induce intended biological responses can also be incorporated into the device. The combination of bioactive encapsulations and PEDOT:PSS electrodes is expected to facilitate favorable biological interactions and electrical performance of the device. Nevertheless, realization of such devices remains challenging due to the high temperature and harsh solvent treatment required for many PEDOT:PSS formulations. Such treatments can irreversibly impair structural integrity and biochemical activity of many bioactive materials, especially those derived from or inspired by mammalian extracellular matrix (ECM). To circumvent this mismatch, one strategy is to cast bioactive materials around a patterned electrode as the last step. However, the non-conducting bioactive layer can potentially interfere with electrical performance of the device. Therefore, novel methods for mild temperature additive manufacturing of highly conducting PEDOT:PSS hydrogels and patterning of bioactive encapsulations are presented.

### SUMMARY OF THE INVENTION

Among the various aspects of the present disclosure are the provision of a bioelectronic device, additive manufacturing methods of bioactive material encapsulated conducting polymer hydrogel electrodes, and related methods of use.

In one aspect, a 3D-printed bioelectronic electrode device is disclosed. The device comprises a conducting polymer hydrogel with a conductivity of a least 2000 S/m and a water fraction ranging from about 70% to about 99.999%, the conducting polymer hydrogel directly patterned on bioactive materials or operatively coupled in electrical contact with a metal or metal oxide substrate functionalized with bioactive materials, their related factors, and any combination thereof. In some aspects, the conducting polymer comprises PEDOT:PSS. In some aspects, the substrate is selected from gelatin, collagen, elastin, ECM protein, polysaccharide, and any combination thereof. In some aspects, biological cells are integrated into the device. In some aspects, the metal or metal oxide contact is surface treated with a thiol or silane terminated compound to enhance adhesion of the conducting polymer hydrogel to the metal or metal oxide surface. In some aspects, the surface treatment compound is cysteamine or (3-aminopropyl)trimethoxysilane. In some aspects, the device is configured to perform the functions of a cardiac patch, nerve cuff, bone growth stimulator, smart bandage for wound healing, surface electrode for ECG or ECoG recording, and any combinations thereof. In some aspects, the device is configured to perform an electrochemical impedance spectroscopy in vitro experiment, the in vitro experiment designed to perform impedance-based monitoring of cell behaviors, the cell behaviors including but not limited to attachment, proliferation, death, barrier properties, contraction, and any combination thereof.

In another aspect, a method to fabricate a bioelectronic device is disclosed. The method comprises depositing a bioactive material surrounding at least one electrode, the deposition comprising: 3D ink printing a conducting polymer onto a substrate; patterning a removable sacrificial material onto the substrate; depositing a cast solution comprising a solvent and bioactive materials and factors on top of the functionalized substrate; solidifying the bioactive material through gelation or evaporating the cast solution solvent; and removing the sacrificial material to expose at least one electrode through the bioactive material. In some aspects, the substrate comprises a second bioactive material and the method further comprises depositing the at least one electrode onto the substrate prior to the deposition of the bioactive material or depositing the at least one electrode within the spaces produced by the removal of the sacrificial material after the removal of the sacrificial material. In some aspects, the 3D ink printing of the conducting polymer is performed at a room temperature ranging from about 20° C. to about 25° C. In some aspects, the sacrificial material is performed at a temperature ranging from about 25° C. to about 55° C. In some aspects, the method further comprises surface-treating a metal or metal oxide contact with and a thiol or silane terminated compound in order to enhance adhesion to the electrode surface. In some aspects, the conducting polymer is PEDOT:PSS. In some aspects, the substrate is selected from gelatin, collagen, elastin, any other ECM protein, polysaccharide, and any combination thereof. In some aspects, the removable sacrificial material is selected from a lipid, lipid composite, chocolate, white chocolate, cacao butter, coconut oil, and any combination thereof. In some aspects, the method further comprises soaking the fabricated device in 70% ethanol after the fabrication process.

In another aspect, a micro-scale 3D-printed material is disclosed that comprises a lipid or lipid composite configured to be deposited in micro-scale 3D structures onto a biocompatible substrate and further configured to be removed from the substrate with material processing at a temperature ranging from about 25° C.-55° C. In some aspects, the material is composed of chocolate, white chocolate, cacao butter, coconut oil, and any combination thereof. In some aspects, the biocompatible substrate is selected from gelatin, collagen, elastin, any other ECM protein, polysaccharide, and any combination thereof.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a schematic showing additive manufacturing of conducting polymer electronics with a bioactive substrate and encapsulation. A conducting polymer or its mixture is directly patterned onto a bioactive substrate, followed by the deposition of sacrificial materials for templating the top encapsulation. The top encapsulation layer is formed as bioactive materials solidify through means such as solvent evaporation or gelation. After removal of the sacrificial material, designated openings are created for direct interfacing with the target system or connecting to instruments.

FIG. 2A is a graph of the conductivity of cast hydrogels (with 40 mg/mL ionic liquid) increased with decreased thickness. N≥3. Mean and standard deviation presented.

FIG. 2B is a set of representative images of PEDOT:PSS hydrogels (with 40 mg/mL ionic liquid) cast at different thickness. Hydrogels that are stable in aqueous environments are formed when cast as a thin layer (thickness≤1000 μm).

FIG. 2C is a pair of images showing that with the identified additives, PEDOT:PSS can be deposited through inkjet printing. Depending on the specific ink formulation, post-treatment such as soaking in 70% ethanol was applied to the dried samples. The resulting prints form stable conducting hydrogel patterns on bioactive substrates with 3 v/v % dodecylbenzenesulfonic acid. Representative images of PEDOT:PSS hydrogel patterns on collagen substrate.

FIG. 2D is a pair of images showing that with the identified additives, PEDOT:PSS can be deposited through extrusion printing. Depending on the specific ink formulation, post-treatment such as soaking in 70% ethanol was applied to the dried samples. The resulting prints form stable conducting hydrogel patterns on bioactive substrates with 40 mg/mL ionic liquid. Representative images of PEDOT:PSS hydrogel patterns on collagen substrate.

FIG. 3A is an image of showing that fat-based materials such as cacao butter and its composites are extruded at mild temperatures and built into 3D structures. Representative image of printed white chocolate pillars on PEDOT:PSS electrode with collagen substrate.

FIG. 3B is an image of a millimeter scale cacao butter pillar with high aspect ratio.

FIG. 3C is an image showing that due to the solvent compatibility and mild removal conditions of these pillars, the fat based sacrificial material issued for patterning solvent cast collagen film, as well as creating 3D channels in cast polydimethylsiloxane.

FIG. 3D is an image wherein the channel was filled with colored water to indicate successful removal of the sacrificial material.

FIG. 4 is a schematic of additive manufacturing of conducting polymer electronics partially encapsulated with bioactive materials. It shows a conducting polymer, or its mixture being directly patterned onto metal or metal oxide electrodes, then sacrificial materials are deposited to template the cast bioactive top encapsulation. After the bioactive encapsulation solidifies, the sacrificial materials are removed to create designated openings for direct interfacing with the target system.

FIG. 5A is a schematic of the disclosed network of PEDOT:PSS/PEGDE hydrogel adhered to cysteamine treated gold substrate. When PEDOT:PSS/PEGDE is deposited on the cysteamine treated gold, it is thought that there is a reaction between the amine group of cysteamine and the epoxy rings in PEGDE, which in turn anchors the PEDOT:PSS/PEGDE network to the gold substrate.

FIG. 5B is an image of the attached samples that remained stable in aqueous conditions for more than 90 days (samples were not further monitored).

FIG. 6A is a representative image of an in vitro impedance sensing device with collagen encapsulation and PEDOT:PSS (with 3 v/v % PEGDE) electrodes.

FIG. 6B is an image showing how the device has been used to measure impedance of human umbilical vein endothelial cell culture over 14 days. Image showing cells stained with LIVE/DEAD™ (Calcein AM; green/ethidium homodimer-1; red), and Hoechst (blue) on day 14. Area circled in white dotted lines indicates the PEDOT:PSS electrode.

FIG. 6C is a representative graph of impedance of a single electrode measured at 4,000 Hz over 14 days. Impedance increased as the cell layer expanded and matured.

FIG. 7A is a schematic diagram illustrating the use of collagen as a bioelectronic encapsulation material to generate amino acids or other components that can be absorbed and used by the body when degraded.

FIG. 7B is a schematic diagram illustrating the use of collagen and other extracellular matrix (ECM) components as a bioelectronic encapsulation material to actively support cell proliferation and tissue remodeling.

### DETAILED DESCRIPTION OF THE INVENTION

The present disclosure is based, at least in part, on the discovery that bioelectronic electrodes with high conductivity and high water fraction can be fabricated with mild processing conditions. As shown herein, compositions, as well as methods of fabrication and methods of use, of bioelectronic hydrogel electrodes are described.

The present teachings include a 3D-printed bioelectronic electrode device. In some aspects, the device can include a conducting polymer hydrogel with a conductivity of >2000 S/m and a high water fraction in contact with a substrate directly fabricated, functionalized, or coated with natural or synthetic ECM components and/or other biological factors. In another aspect, the conducting polymer can be PEDOT:PSS. In accordance with another aspect, the substrate can be selected from gelatin, collagen, elastin, any other ECM protein, polysaccharide, or non-biological polymers functionalized with ECM or ECM-mimicking peptides, and any combination thereof. In yet another aspect, the device can be configured to perform the functions of an electric cell substrate impedance sensing electrode, a microelectrode array, surface electrode, cardiac patch, nerve cuff, bone growth stimulator, smart bandage for wound healing, and any combination thereof.

The present teachings also include a method to fabricate a bioelectronic device. In one aspect, the method can include 3D ink printing a conducting polymer onto a substrate. In another aspect, the method can include patterning a sacrificial material that is removable under physiological conditions onto the substrate. In another aspect, the method can include depositing a cast solution comprising a solvent, water, and bioactive materials and factors on top of the functionalized substrate for top encapsulation. In accordance with another aspect, the method can include casting with or without evaporating the cast solution solvent. In yet another aspect, the method can include removing the sacrificial material to create electrical openings in the bioactive encapsulation. In another aspect, the method can include depositing a conducting material on the exposed substrate locations where the sacrificial material was removed to fabricate electrodes or electronic contacts. In another aspect, the method can be performed at room temperature. In an aspect, the conducting polymer can be PEDOT:PSS. In another aspect, the substrate can be selected from gelatin, collagen, elastin, any other ECM protein, polysaccharide, and any combination thereof. In yet another aspect, the physiological removable sacrificial material can be selected from a fat, fat composite, chocolate, white chocolate, cacao butter, coconut oil, and any combination thereof.

The present teachings also include a method to fabricate a bioelectronic device that includes depositing electrical contact pads onto a substrate, 3D printing a conducting polymer onto the contact pads, patterning a physiological removable sacrificial material on top of the conducting polymer, depositing a cast solution that can include a solvent, and bioactive materials and factors on top of the functionalized substrate, evaporating the cast solution solvent, and removing the physiological removable sacrificial material, exposing the patterned conducting polymer on the substrate. In one aspect, the method can be performed at room temperature. In another aspect, the conducting polymer can be PEDOT:PSS. In yet another aspect, the substrate can be selected from gelatin, collagen, elastin, any other ECM protein, polysaccharide, and any combination thereof. In yet another aspect, the physiological removable sacrificial material can be selected from a lipid, lipid composite, chocolate, white chocolate, cacao butter, coconut oil, and any combination thereof.

The present teachings also include methods of performing in vitro and in vivo electrochemical impedance spectroscopy (EIS), electrical stimulation and/or recording. By way of non-limiting example, the disclosed methods may be used to perform a bioelectronics experiment. For in vitro applications, the disclosed electrode can serve as a more bioactive and cytocompatible alternative to commercially available EIS devices for monitoring various cell behaviors including but not limited to attachment, proliferation, mobility, barrier properties, and contraction. In addition, the disclosed electrode can also serve as microelectrode arrays (MEA) to record field potentials of electroactive cells (neurons, cardiomyocytes). In some aspects, the disclosed electrode can also be used to stimulate specific cell types to promote proliferation and differentiation, and to induce electrical responses.

For in vivo applications, the disclosed electrode can be used to record electrical signals such as ECG and ECoG. In some aspects, the disclosed electrode can be used to apply electrical stimulation, which has been established to promote tissue regeneration, especially for peripheral nerve injury, bone growth, and wound healing applications. The bioactive encapsulation would have added benefits since the material itself can promote regeneration.

In one aspect, the method can include integrating cells on the bioactive encapsulation of the bioelectronic device as described herein, interfacing cells with the PEDOT:PSS electrode of the device, or implanting the bioelectronic device into a living organism and performing a bioelectronics experiment.

In one aspect, the disclosed electrode can be used to record electrical signals such as ECG and ECoG to diagnose and/or monitor a disorder in a patient in need. In another aspect, the disclosed electrode can be used to treat a disorder including, but not limited to, a bioelectronics-related disorder in a subject with electrical stimulation. In some aspects, the method can include implanting the bioelectronic device as described in the present disclosure into the subject and stimulating the electrodes with at least one electrical impulse. In other aspects, the bioelectronic-related disorder can be selected from a neural disease, nerve disease, cardiac disease, bone disease, wound, and any combination thereof. In yet another aspect, the bioelectronic device can be configured to perform the functions of a cardiac patch, nerve cuff, bone growth stimulator, smart bandage for wound healing, and any combination thereof.

One aspect of the present disclosure provides PEDOT:PSS inks and treatments that enable room temperature gelation after direct deposition on bioactive substrates. PEDOT:PSS aqueous dispersion by itself, or mixed with gelation agents, which include but are not limited to at least one acidic surfactant such as dodecylbenzene sulfonic acid (DBSA), ionic liquid (IL) such as 4-(3-Butyl-1-imidazolio)-1-butanesulfonic acid triflate, salt such as NaCl, and polymers such as poly(ethylene glycol) diglycidyl ether (PEGDE) and poly(ethylene glycol) thiol (PEGSH) is deposited with a thickness of 1 mm or thinner. Due to the variety of inks developed, the present disclosure can satisfy the requirement of numerous fabrication techniques including drop casting, molding, extrusion-based printing, and inkjet printing with respective ink formulations. Depending on the substrate and gelation agent, the solution can either form a stable hydrogel after sitting at room temperature, or through the treatment with a mixture of water and a non-solvent solution, which includes but is not limited to ethanol, isopropanol, acetone, and other solutions that do not dissolve PEDOT. After soaking in deionized water (DIH2O), the resulting hydrogel has a >90% water fraction. When deposited on dry substrates such as glass or dry ECM films, which include but are not limited to collagen, albumin, and hyaluronic acid, the fully formed PEDOT:PSS hydrogel exhibits kS/m range conductivity. In addition, the ink can also be directly deposited on a hydrogel substrate, which includes but is not limited to gelatin, collagen, elastin, and other ECM proteins or polysaccharide-based hydrogels, and can form a water-stable conducting hydrogel.

In some aspects, the conducting polymer hydrogel can have a water fraction of at least 70%. In other aspects, the water fraction can be at least 80%. In yet other aspects, the conducting polymer hydrogel can have a water fraction of at least 90%. In accordance with another aspect, different processing conditions can impact water fraction of the hydrogels. In yet other aspects, fabrication can occur under mild processing conditions. In yet other aspects, processing conditions can include temperature, solvent composition, pH, and water conditions. In some aspects, the temperatures employed in fabrication are below the denaturation temperature of the molecules used in the processing. In other aspects, patterning can occur below 40° C. In another aspect, collagen deposition can occur below 65° C.

The versatility of the inks, in combination with its facile fabrication procedure and readily available reagents, make it highly suitable for commercialization. For example, one of our PEDOT:PSS patterning procedures only requires the deposition of a commercially available PEDOT:PSS dispersion by itself, drying of the print, and treatment in 70% ethanol. Such straightforward, low-cost procedures can be easily adapted for industrial production.

To pattern the top encapsulation with an additive manufacturing approach, a physiological temperature removable sacrificial material can be utilized to create an electrode opening in the bioactive encapsulation. To avoid dehydration and change in the dimension of the sacrificial material, fat and fat composites including, but not limited to, cacao butter, coconut butter, Crisco, or chocolate can be utilized for patterning solvent cast films. Once the top encapsulation is fully formed, the sacrificial material can be easily removed by soaking the material in warm water at a temperature of less than about 40° C. If the top encapsulation is easy to extrude (e.g. gelatin hydrogel), it can also be directly deposited and patterned onto the conducting material and substrate.

The use of fat or fat composite as sacrificial materials is a unique aspect of the present disclosure that has not been previously reported. In addition, the fat-based sacrificial materials were also found to be stable in numerous organic solvents including dimethylsulfoxide (DMSO) and acetone. Thus, the present disclosure can be easily applied to patterning a variety of solvent-cast films with aqueous or organic solutions. Besides films, these fat-based sacrificial materials were also successful in creating channels in 3D objects, such as polydimethylsiloxane (PDMS) microfluidics, which are commonly fabricated with photolithography or thermoplastic sacrificial materials. With the fat-based sacrificial material, a device with 3D open channels can be easily fabricated by casting PDMS over extruded channel positives, letting it cure, and removing the sacrificial material with warm water.

Depending on the intended application and device design, the completed device can be partially or fully encapsulated with bioactive materials beside openings for the electrode. Additional insulative materials may be added between the conducting hydrogel and the encapsulation to further enhance device performance. A rigid, inorganic substrate such as glass, gold, or indium tin oxide may be used to carry the device or enable electrical connection for in vitro applications, while the soft and hydrated components are directly interfacing with the cells. For both in vitro and in vivo applications, metal connectors and wiring may be used to connect the device to an external controller. With bioactive encapsulation, the present disclosure can be applied to numerous existing research or therapeutic devices for improved biointerfacing. Potential in vitro applications may include multi-electrode arrays (MEAs) for stimulating or recording electroresponsive cells, impedance measurements to evaluate cell growth or barrier functions, or applying electrical fields to enhance stem cell differentiation. Potential in vivo applications may include but are not limited to implantable or external devices such as cardiac patches, nerve cuffs, bone growth stimulators, smart bandages for wound healing, or surface electrodes for ECG or ECoG recording.

Compared to limited existing methods of forming PEDOT:PSS hydrogels at room temperature, the methods of the current disclosure are unique due to the following reasons: 1) this disclosure is the first to demonstrate the compatibility of patternable PEDOT:PSS inks to mammalian ECM substrates and vice versa. On certain ECM substrates, not all PEDOT:PSS precursors can readily form a water-stable hydrogel. Post-treatment methods were tested to counter this problem. While not all existing post-treatments for stand-alone PEDOT:PSS or PEDOT:PSS patterning on inorganic substrates are effective, it was determined that several post-treatment methods that result in similar conductivities of PEDOT:PSS hydrogel on ECM substrates compared to those patterned on glass or other in organic substrates; 2) the method is highly adaptable to other substrates or gelation agents. The underlying principle is to deposit the ink as a thin layer and use either an excess of gelation agent or a post-print treatment; 3) the resulting pure PEDOT:PSS hydrogels exhibit both high water fraction and high conductivity (kS/m range), while all existing methods of room temperature gelled PEDOT:PSS reported <90% water fraction. The high water fraction of the PEDOT:PSS hydrogel gives it tissue-like properties that can create a favorable soft and hydrated microenvironment for surrounding cells, thus making it more suitable for biointerfacing applications; 4) the inks can be directly 3D printed via extrusion or ink jetting onto a substrate using established, commercially available 3D printing set up. These printing configurations are not restricted to the 3D printer used for testing, and can be adapted to other commercial 3D printers capable of jetting or extrusion printing; 5) the ink can be easily formulated with commercially available PEDOT:PSS aqueous dispersion, without the need for multistep processing and concentrating the PEDOT:PSS prior to use.

In some aspects, the present teachings include a description of bioactive materials. Bioactive materials are defined as materials that are intentionally employed to induce or modulate biological activities to enhance a targeted function. Materials that are derived from, inspired by the native extracellular matrix (ECM), as well as other natural and synthetic biomaterials loaded or functionalized with drugs and growth factors, can all be utilized as bioactive materials. Functional motifs on bioactive materials are often sensitive to harsh conditions, such as high temperatures, extreme pH levels, and incompatible solvents. Therefore, the methods and approaches described in this application is expected to be applicable to a broad range of potential bioactive materials. In specific discussions regarding collagen film as the substrate or encapsulation, use of “mammalian ECM” can be used. In other aspects, non-mammalian natural polymers such as silk fibroin can be used.

In some aspects, additional additives have been identified that can facilitate room temperature gelation of PEDOT:PSS on collagen substrates. The first additive is poly(ethylene glycol) diglycidyl ether, which has been reported to crosslink PEDOT:PSS at room temperature. However, it has not been used in combination with a bioactive substrate or encapsulation.

The present teachings also describe a developed in vitro device with top collagen encapsulation and metal or metal oxide connections (gold, platinum, indium tin oxide, etc.) patterned on a smooth substrate (glass, polyimide, polycarbonate, etc.). Unlike the fully encapsulated device, which has a penetrable polymer network in its substrate that allows stable adhesion of PEDOT:PSS electrode, the partially encapsulated device requires additional treatment to facilitate PEDOT:PSS adhesion. A method has been developed that involves first treating noble metal substrates (e.g. gold or platinum) with cysteamine. When PEDOT:PSS/PEGDE is deposited, reaction between the amine group of cysteamine and the epoxy rings in PEGDE enables stable adhesion of the PEDOT:PSS/PEGDE network to the noble metal substrate. Different concentrations of PEGDE were tested, and the attached samples remained stable in aqueous conditions for more than 90 days (samples were not further monitored). This method can be adopted for different substrates and formulations by treating the substrate with a molecule that forms stable connection to the substrate on one end, and reacts with PEDOT:PSS or its additives on the other end (e.g. thiol or silane terminated surface treatment with amine or other functional groups that reacts with epoxy). There is no prior report of the use of cysteamine in combination with PEDOT:PSS/PEGDE hydrogel.

The present teachings also demonstrate the feasibility of the use of a partially collagen encapsulated conducting polymer device for in vitro impedance monitoring of cell culture. The impedance of cell culture was measured for 14 days. In some aspects, the device can be used in in vitro electrochemical impedance spectroscopy. The device can be used to perform impedance-based monitoring of cell behaviors including but not limited to attachment, proliferation, death, barrier properties, and contraction.

In other aspects, the teachings make use of fat-based sacrificial material, which have versatile properties. This class of materials satisfies several requirements for sacrificial materials all at once: 1) stability in aqueous solution or hydrogels 2) ease of removal with mild treatment 3) resistance to evaporation. All three properties listed above have not been simultaneously met by other sacrificial materials such as hydrogels (change dimensions when dehydrated), thermoplastic (requires high temperature or harsh solvent to remove), or carbohydrate glass (soluble in aqueous solution). Besides patterning electrode openings for the device described in this application, fat-based sacrificial materials can also be used to create channels in polydimethylsiloxane (PDMS). Therefore, it is envisioned that this approach can have additional applications beyond fabrication of the above device described.

In yet more aspects, not all of the steps of the fabrication process need to be carried out at room temperature. The sacrificial materials may require slightly elevated temperature for their deposition and removal (e.g. 34-38° C. for cacao butter). This temperature is still considered mild and acceptable for processing in conjunction with bioactive materials.

### Bioelectronic Modulation Devices

As described herein, bioelectronics signals have been implicated in various diseases, disorders, and conditions. As such, modulation and recording of bioelectronic signals (e.g., modulation of a bioelectronic neural signal) can be used for the treatment of such conditions. A bioelectronic modulation device can modulate bioelectronic response or induce or inhibit bioelectronic signals. Bioelectronic modulation can comprise modulating bioelectronic signals produced or received by cells, or modulating the quality of the bioelectronic signal produced or received by cells.

Bioelectronic modulation devices may include any of the substrates, hydrogels, and other structures disclosed herein that can modulate bioelectronic signals produced or received by cells (e.g., inducing a bioelectronic signal with a cardiac patch, nerve cuff, bone growth stimulator, or smart bandage for wound healing). For example, a bioelectronic modulation device can be an activator, an inhibitor, an agonist, or an antagonist.

In various aspects, the present disclosure provides methods of treating or preventing an adverse cardiac event, a neuronal disorder, a bone disease, or a wound using, by way of non-limiting example, a high conductivity and large water fraction hydrogel electrode that is fabricated at room temperature as described herein.

### Therapeutic Methods

Also provided is a process of treating, preventing, or reversing a disease or disorder associated with bioelectronic signals in a subject in need of administration of a bioelectronics device configured to provide bioelectronic signals as described herein, to treat a disease associated with biological signals, or the lack thereof. Non-limiting examples of diseases or disorders that can benefit from electrical stimulation include an adverse cardiac event, a neuronal disorder, nerve or bone injury, or a wound. By way of non-limiting example, the bioelectronics device used for treatment may include a high conductivity and large water fraction hydrogel electrode fabricated at room temperature as described herein.

Methods described herein are generally performed on a subject in need thereof. A subject in need of the therapeutic methods described herein can be a subject having, diagnosed with, suspected of having, or at risk for developing a disease associated with bioelectronic signals, or a lack thereof, or a subject that can benefit from pro-regenerative electrical stimulation. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of bioelectronic signal is, for example, an amount that would cause the desired therapeutic effect in a subject while minimizing undesired side effects. In various embodiments, an effective amount of bioelectronic signal described herein can substantially inhibit a disease associated with bioelectronic signals, or a lack thereof, slow the progress of a disease associated with bioelectronic signals, or a lack thereof, or limit the development of a disease associated with bioelectronic signals, or a lack thereof.

Again, each of the states, diseases, disorders, and conditions, described herein, as well as others, can benefit from the large water fraction hydrogel electrodes and bioelectric devices as described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of bioelectronic signals can occur as a single event or over a time course of treatment. For example, a bioelectronics signal can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for a disease associated with bioelectronic signals, or a lack thereof.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to PEDOT:PSS conducting polymers, collagen, ECM proteins, surfactants such as DBSA, a substrate, ionic liquid, solvent treatment, and water. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Additive Manufacturing of Extracellular Matrix Encapsulated Conducting Polymer Electrodes

To characterize the fabrication of hydrogel electrodes for bioelectronics applications. the following experiments were conducted. Collagen film can be easily fabricated by homogenizing collagen powder with various concentrations of acetic acid and glycerol, casting the slurry, and air drying the slurry into a film. The resulting film is easy to handle and conformable to various underlying surfaces when hydrated. Tensile testing of the hydrated films showed that these films can be elongated to more than 150% of resting length before the hydrated films reach their ultimate strengths. The tensile modulus and strain of each hydrated film at ultimate strength suggest that the collagen films can easily withstand surgical handling and tissue movement without failing.

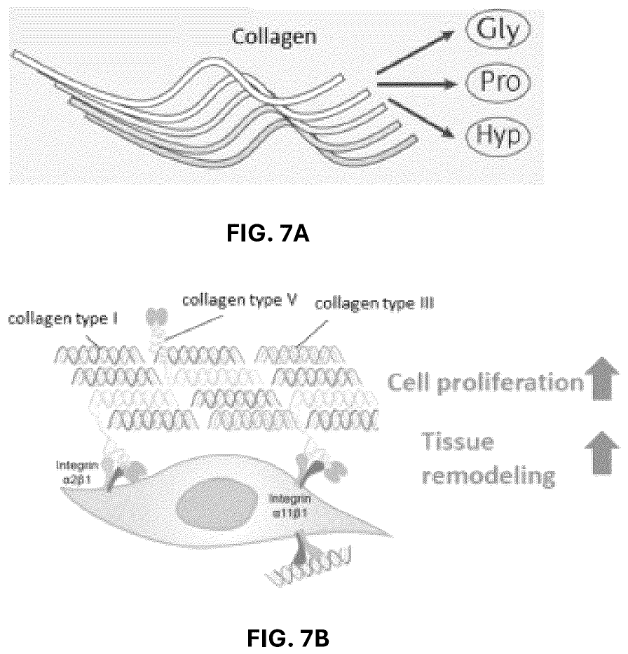

Collagen and other extracellular matrix (ECM) components can actively support cell proliferation and tissue remodeling (FIG. 7B). Moreover, when they degrade, they generate amino acids or other components that can be absorbed and used by the body (FIG. 7A). To demonstrate the cytocompatibility of the collagen films, cultured human adult dermal fibroblasts were cultured on collagen films for up to 28 days. They expanded to confluency and exhibited high viability throughout the culture period. During the beginning of the culture, cells seeded on collagen showed faster expansion as suggested by a higher % increase in cell number compared to tissue culture plastic (TCP) control.

In some aspects, dodecylbenzenesulfonic acid (DSA) is added to the PEDOT:PSS ink composition to facilitate the curing the ink at room temperature conditions when printed onto a variety of substrates to form features with dimensions on the order of hundreds of micrometers. DBSA has been previously used at low concentrations (below 1%) as a surface tension modifier for jetting PEDOT:PSS or as a dopant for PEDOT:PSS films. It should be noted that with a low concentration of DBSA, thermal treatment is needed for PEDOT:PSS to form a water-stable network. It was discovered that PEDOT:PSS ink with a sufficient concentration of dodecylbenzenesulfonic acid (DSA), when applied to a substrate in a user-defined pattern using an inkjet device, forms room temperature-gelled, highly conducting PEDOT:PSS hydrogel tracks. With higher concentrations of DBSA (3-7 wt %), PEDOT:PSS forms stable hydrogels at room temperature. However, this method of casting bulk hydrogels resulted in low conductivity of ˜10 S/m when the gels were hydrated. In contrast, when PEDOT:PSS ink with 3 v/v % DBSA was cast or ink jetted as a thin layer, significantly higher conductivity was obtained while still maintaining a high water fraction.

In some aspects, methods to inkjet a PEDOT:PSS ink with a sufficient concentration of dodecylbenzenesulfonic acid (DBSA) to form room temperature gelled, highly conducting PEDOT:PSS hydrogel tracks are disclosed. PEDOT:PSS with 3% v/v of DBSA inkjet printed on a glass and collagen substrates, respectively, at room temperature, demonstrated a hydrated conductivity of the gelled ink was 3719 S/m and the water fraction is 98.1% The minimum feature size of the inkjet printed gel was 200-300 μm.

Inks with higher concentrations of IL to induce gelation at room temperature have also been developed. When deposited on glass, the ink gels after sitting in ambient conditions. When deposited on collagen, due to the effect of the substrate, a 70% ethanol treatment is required after drying the ink to form a water-stable hydrogel. The room temperature methods gelled PEDOT:PSS on either substrate that exhibited conductivity of 2048 S/m, a water fraction of 94.8%, and minimum feature sizes ranging from about 200 to about 300μ.

To further investigate ink formulations that can form stable hydrogel at room temperature, we cast PEDOT:PSS with various concentrations of IL at different thicknesses and dried at room temperature for 24 hr. While pure PEDOT:PSS cannot form a stable hydrogel by drying alone, PEDOT:PSS with 10 and 20 mg/mL IL formed water stable hydrogel when cast at 500μ thick, and those with 40 and 80 mg/mL IL were able to remain stable at a higher thickness of 1000 μm,

While pure PEDOT:PSS cannot form a stable hydrogel by drying alone, PEDOT:PSS with 10 and 20 mg/mL ionic liquid (IL) formed water-stable hydrogel when cast at 500 μm thickness, and those with 40 and 80 mg/mL IL were able to remain stable at a higher thickness of 1000 μm. This finding brings new insights into the gelation conditions of PEDOT:PSS hydrogels. The formation of hydrogels with a set thickness and one or a few concentrations of gelation agents, as well as the interplay of gel thickness, gelation agent concentration, and temperature demonstrates the PEDOT:PSS forms stable hydrogels at room temperature with suitably high gelation agent concentration and sufficiently low thickness that may be potentially be applied to a variety of different formulations.

This finding brings new insights into the gelation conditions of PEDOT:PSS hydrogel. While previous findings usually report the formation of hydrogels with a set thickness and one or a few concentrations of gelation agents, the interplay of gel thickness, gelation agent concentration, and temperature was not fully addressed. The principle demonstrated here (i.e. PEDOT:PSS can form stable hydrogel at room temperature with a high enough gelation agent concentration and a low enough thickness) can potentially be applied to more formulations in the future.

In addition, it was found that conductivity increases with a decrease in thickness and an increase in IL concentration until it plateaus. When cast at a 500-um thickness, PEDOT:PSS hydrogel with 40 and 80 mg/mL IL respectively got high conductivities of 2165 and 2113 S/m, with no significant difference. Furthermore, it was found that all conditions that resulted in water-stable hydrogels have high water fractions above 94%.

Various post-print treatments were also investigated for inducing PEDOT:PSS ink gelation on collagen substrate. It was found that a blend of 30-50% water and 50-70% ethanol resulted in the highest conductivities (2039 to 3516 S/m) on untreated collagen. Interestingly, a higher concentration of alcohol did not sufficiently induce stable PEDOT:PSS networks. It was also found that crosslinked collagen resulted in more consistent PEDOT:PSS conductivities both dry and hydrated. In addition, 70% ethanol treatment was also able to induce gelation of dried PEDOT:PSS prints without IL on both glass and collagen substrates. A high conductivity of 4398 S/m was achieved by treating PEDOT:PSS printed on collagen.

To confirm the compatibility of the disclosed ink with hydrogel substrates, PEDOT:PSS with 3v/v % DBSA and 40 mg/mL IL were respectively printed on 20 wt % gelatin hydrogel. Both conditions were able to form water-stable PEDOT:PSS hydrogels.

The use of fat or fat composite as sacrificial materials is a unique approach that has not been previously reported. This class of materials satisfies several requirements for sacrificial materials all at once: 1) stability in aqueous solution or hydrogels 2) ease of removal with mild treatment 3) resistance to evaporation. All three properties listed above have not been simultaneously met by other sacrificial materials such as hydrogels (change dimensions when dehydrated), thermoplastic (requires high temperature or harsh solvent to remove), or carbohydrate glass (soluble in aqueous solution).

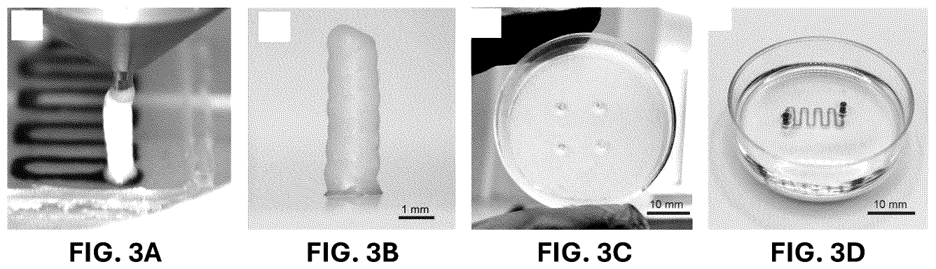

Methods for patterning different kinds of fat and fat composites have been developed (FIGS. 3A, 3B, 3C, and 3D). White chocolate and cacao butter can be deposited into horizontal (serpentine) and vertical (pillars) features via melt extrusion. The pillars can be easily built to a 5 mm height and removed using warm water at 36.5 C. In addition, we also tested the jetting of coconut oil.

With printed chocolate pillars, an opening on collagen films can be created with a step-by-step approach as illustrated in FIGS. 3A, 3B, 3C, and 3D, a set of images showing the templating of collagen films with sacrificial, 3D-printed chocolate. With printed chocolate pillars, openings on collagen films can be easily created using a step-by-step approach. Collagen slurry was cast on chocolate pillars, the collagen slurry was dried into a membrane and after the removal of sacrificial chocolate, open ports remained. This method is intended to create a collagen top encapsulation on substrates that are already patterned with PEDOT:PSS. The use of the sacrificial chocolate material ameliorates several fabrication challenges, including alignment of manual tools used to cut or punch the collagen film; high-temperature denaturation of collagen when using existing sacrificial materials requiring harsh working conditions such as laser cutters; and delamination or attachment of ready-made collagen films on a substrate.

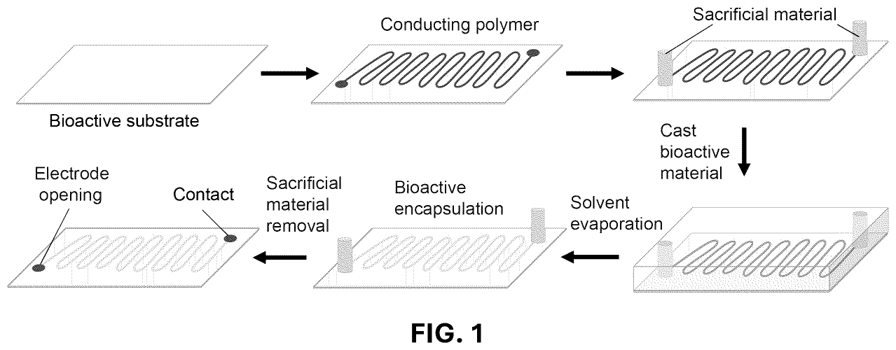

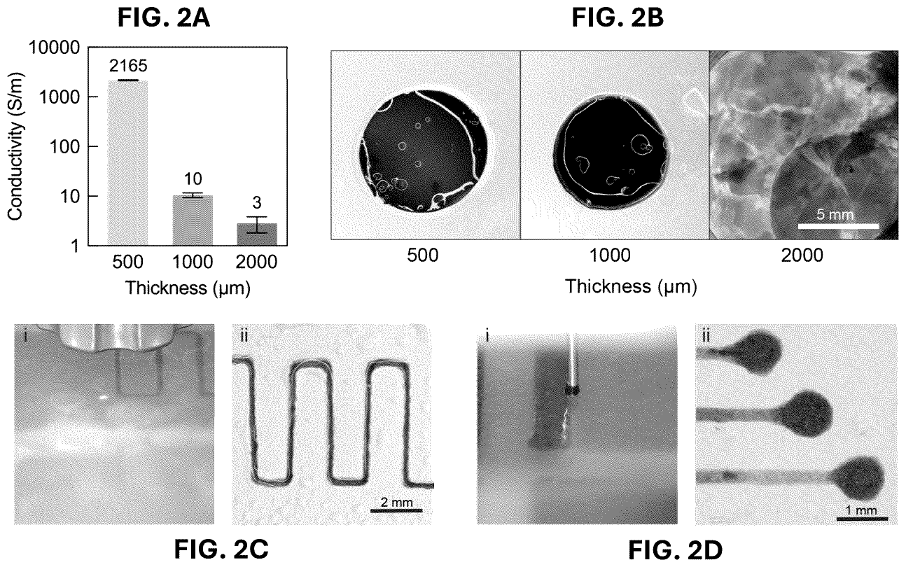

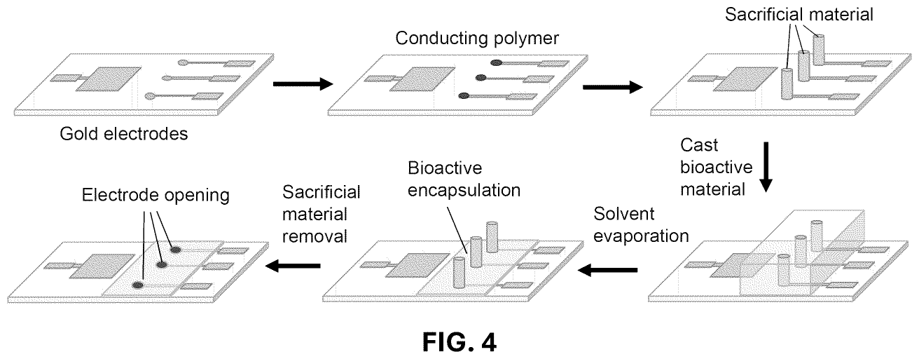

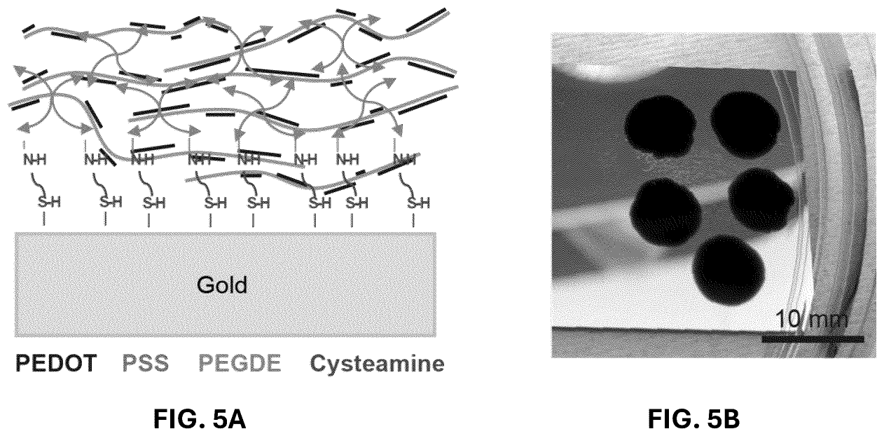

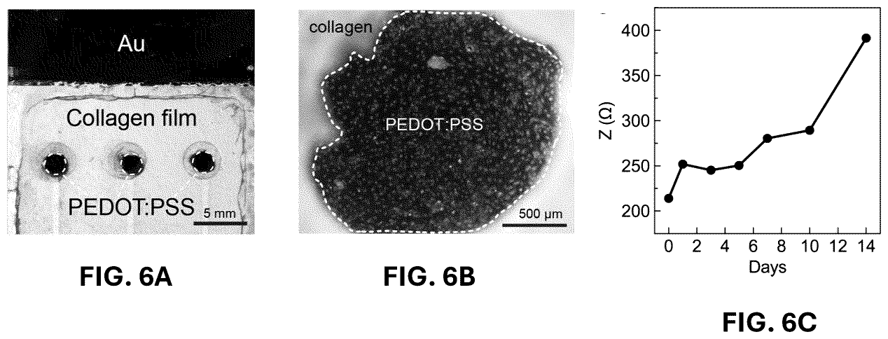

In addition to solvent cast films, 3D-printed fat can also be used to pattern other materials such as PDMS. Working microfluidic channel (FIG. 3D) was created after the removal of the sacrificial chocolate. A schematic of a layer-by-layer fabrication method of an implantable electrode fully encapsulated in collagen can be seen in FIG. 1, and a layer-by-layer fabrication method of a cell culture electrode with collagen top encapsulation can be seen in FIG. 4.

What is claimed is:

1. A 3D-printed bioelectronic electrode device, the device comprising a conducting polymer hydrogel with a conductivity of a least 2000 S/m and a water fraction ranging from about 70% to about 99.999%, the conducting polymer hydrogel directly patterned on bioactive materials or operatively coupled in electrical contact with a metal or metal oxide substrate functionalized with bioactive materials, their related factors, and any combination thereof.

2. The device of claim 1, wherein the conducting polymer comprises PEDOT:PSS.

3. The device of claim 1, wherein the substrate is selected from gelatin, collagen, elastin, ECM protein, polysaccharide, and any combination thereof.

4. The device of claim 1, wherein biological cells are integrated into the device.

5. The device of claim 1, wherein the metal or metal oxide contact is surface treated with a thiol or silane terminated compound to enhance adhesion of the conducting polymer hydrogel to the metal or metal oxide surface.

6. The device of claim 5, wherein the surface treatment compound is cysteamine or (3-aminopropyl)trimethoxysilane.

7. The device of claim 1, wherein the device is configured to perform the functions of a cardiac patch, nerve cuff, bone growth stimulator, smart bandage for wound healing, surface electrode for ECG or ECoG recording, and any combinations thereof.

8. The device of claim 1, wherein the device is configured to perform an electrochemical impedance spectroscopy in vitro experiment, the in vitro experiment designed to perform impedance-based monitoring of cell behaviors, the cell behaviors including but not limited to attachment, proliferation, death, barrier properties, contraction, and any combination thereof.

9. A method to fabricate a bioelectronic device, the method comprising depositing a bioactive material surrounding at least one electrode, the deposition comprising:
a. 3D ink printing a conducting polymer onto a substrate;
b. patterning a removable sacrificial material onto the substrate;
c. depositing a cast solution comprising a solvent and bioactive materials and factors on top of the functionalized substrate;
d. solidifying the bioactive material through gelation or evaporating the cast solution solvent; and
e. removing the sacrificial material to expose at least one electrode through the bioactive material.

10. The method of claim 9, wherein the substrate comprises a second bioactive material and the method further comprises depositing the at least one electrode onto the substrate prior to the deposition of the bioactive material or depositing the at least one electrode within the spaces produced by the removal of the sacrificial material after the removal of the sacrificial material.

11. The method of claim 9, wherein the 3D ink printing of the conducting polymer is performed at a room temperature ranging from about 20° C. to about 25° C.

12. The method of claim 9, wherein the removal of the sacrificial material is performed at a temperature ranging from about 25° C. to about 55° C.

13. The method of claim 9, the method further comprising surface-treating a metal or metal oxide contact with and a thiol or silane terminated compound in order to enhance adhesion to the electrode surface.

14. The method of claim 9, wherein the conducting polymer is PEDOT:PSS.

15. The method of claim 9, wherein the substrate is selected from gelatin, collagen, elastin, any other ECM protein, polysaccharide, and any combination thereof.

16. The method of claim 9, wherein the removable sacrificial material is selected from a lipid, lipid composite, chocolate, white chocolate, cacao butter, coconut oil, and any combination thereof.

17. The method of claim 9, the method further comprising soaking the fabricated device in 70% ethanol after the fabrication process.

18. A micro-scale 3D-printed material, the material comprising a lipid or lipid composite configured to be deposited in micro-scale 3D structures onto a biocompatible substrate and further configured to be removed from the substrate with material processing at about 25-55° C.

19. The material of claim 18, wherein the material comprises chocolate, white chocolate, cacao butter, coconut oil, and any combination thereof.

20. The material of claim 18, wherein the biocompatible substrate is selected from gelatin, collagen, elastin, any other ECM protein, polysaccharide, and any combination thereof.
